(19) United States
(12) Patent Application Publication
(10) Pub. No.: US 2009/0222051 A1
(43) Pub. Date: Sep. 3, 2009

(54) SPS - FB BONE PLATE SCREW FIXATION SYSTEM

(76) Inventor: Jose Roberto PENGO, Sao Paulo (BR)

Correspondence Address: LADAS & PARRY LLP, 224 SOUTH MICHIGAN AVENUE, SUITE 1600, CHICAGO, IL, 60604, US

(21) Appl. No.: 12/039,788

(22) Filed: Feb. 29, 2008

(51) Int. Cl. A61B 17/58

(52) U.S. Cl. 606/305; 606/301

(57) ABSTRACT

The present patent of invention that refers to a bone plate screw fixation. It refers to a system comprising a screw (3) and a plate (1) for bone synthesis, wherein the screw (3) after being placed in the bone in the final tight will stay blocked on the plate (1) whether using the lock ring (2) or not. On A Version (FIG. 1) the system will be comprised by screw (3), plate (1) and lock ring (2). The screw (3) has its conical head that may receive a thread or not. The ring (2) has in its internal outline a cone that may have a thread to receive the screw (3) or not. Outside the lock ring (2) there is a spherical surface that will connect to the plate (1), which will fix the set when tightened; this ring is cut so that it can be expanded. The plate (1) has its spherical hole similar to ring's outward to receive the set (2) and (3) where the block will occur. However, on B Version (FIG. 2) the screw (3) may be placed on the plate (5) even without the lock ring following the screw (3) thread outline (4).

The present patent of invention refers to a new industrial process of bone plate screw fixation system to which original construction was given aiming at product usage and efficiency.

It refers to a system used in all kind of “Bone Synthesis”, which are implants that suits for bone fixation so that fracture consolidation can be made, composed of several components such as screw for fixation of the plate with conical head that may receive or not a thread, this plate will fix the set (screw/plate/bone/ring) when tight and a lock ring having a cone in its internal outline which may or may not have a thread to receive the screw.

One inconvenient that may be a problem for this type of invention is the fact that conventional systems of plate/screw bone fixation do not allow screw fixation on the plate what may cause system dismantling throughout time, mainly in more porous bones.

Having in mind this problem and aiming at overcoming it, the System “SPS—FB Bone Plate Screw Fixation System” object of the present invention was developed, whose original construction and industrial application was possible due to a new bone plate screw fixation system.

### BRIEF DESCRIPTION OF THE DRAWING FIGURES

For a better understanding of the present invention here it follows a detailed description of it with reference to the accompanying drawings wherein the figures depicts an original form of bone plate screw fixation system capable of providing better quality and safety in this system, in which:

FIG. 1 shows a perspective view of the screw being fixed to the plate; and

FIG. 2 presents a perspective view of the screw placed on the plate without the lock ring following screw thread outline.

### DETAILED DESCRIPTION OF THE PREFERRED EMBODIMENT

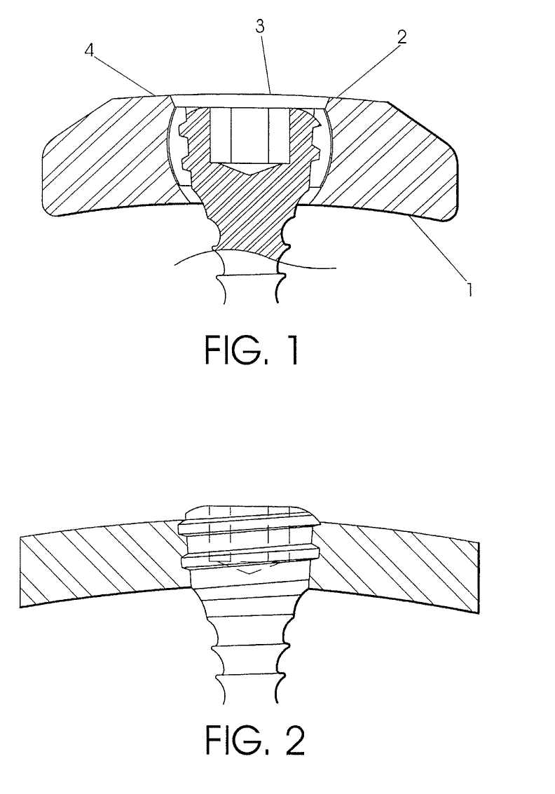

According to the drawings the present patent of invention “SPS—FB BONE PLATE SCREW FIXATION SYSTEM” characterizes by comprising a bone plate screw fixation system (FIG. 1) comprised by a screw (3) for fixation in the plate (1) becoming a rigid set of screw/plate/bones/lock ring (2) that allows the screw (3) to be introduced in different positions in the bone (convergent and divergent).

The device comprises screw blocking (3) in the plate (1) for performing (bone synthesis) the screw having absolute freedom of position (3) during surgery.

It may be used in any implant for bone synthesis with any implant system and indication already known. It may be done in any biocompatible material.

It refers to a system comprised by screw and plate for bone synthesis wherein the screw (3) after being placed in the bone in the last tight becomes blocked on the plate (1) whether using the lock ring or not (2).

The system is presented in two versions, being A Version (FIG. 1) with lock ring (2). The system will be comprised by three basic elements: screw (3), plate (1) and lock ring (2). The screw (3) has its conical head what may receive a thread or not. The ring (2) has a cone in its internal outline that may have a thread to receive the screw (3) or not. Outside the lock ring (2) there is a spherical surface that will connect to the plate (1), which will fix the set when tightened: this ring is cut so that it can be expanded. The plate (1) has its spherical hole similar to ring's outward to receive the set (2) and (3) where the block will occur. However, on B Version (FIG. 2) the screw (3) may be placed on the plate (5) even without the lock ring (FIG. 1) (2) following the screw (3) thread outline (4).

Finally, the present patent of invention presents a new original and functional form to meet this kind of products users' needs.

1. “SFP—FB BONE PLATE SCREW FIXATION SYSTEM” comprised by industrial process for bone plate screw fixation to which was given original construction, characterized by being comprised by a screw (3) and a plate (1) for bone synthesis wherein the screw (3) after being placed in the bone in the last tight will be blocked on the plate (1) whether using or not the lock ring (2). On A Version (FIG. 1) the system will be comprised by three basic elements: screw (3), plate (1) and lock ring (2). The screw (3) has its conical head that may receive a thread or not. The ring (2) has in its internal outline a cone that may have a thread to receive the screw (3) or not. Outside the lock ring (2) there is a spherical surface that will connect to the plate (1), which will fix the set when tightened; this ring is cut so that it can be expanded. The plate (1) has its spherical hole similar to ring's outward to receive the set ring (2) and screw (3) where the block will occur. However, on B Version (FIG. 2) the screw (3) may be placed on the plate (5) even without the lock ring following the screw (3) thread outline (4).
